(19) United States
(12) Patent Application Publication
(10) Pub. No.: US 2022/0240765 A1
(43) Pub. Date: Aug. 4, 2022

(54) ENDOSCOPE FORCEPS LIFTING DEVICE, ENDOSCOPE HEAD PART AND DUODENOSCOPE SYSTEM

(71) Applicant: SONOSCAPE MEDICAL CORP., Shenzhen, Guangdong (CN)

(72) Inventors: Feng JING, Shenzhen, Guangdong (CN); Keduan XU, Shenzhen, Guangdong (CN); Gongan WU, Shenzhen, Guangdong (CN); Pan LI, Shenzhen, Guangdong (CN); Zican XIANG, Shenzhen, Guangdong (CN)

(73) Assignee: SONOSCAPE MEDICAL CORP., Shenzhen, Guangdong (CN)

(21) Appl. No.: 17/622,721

(22) PCT Filed: May 26, 2020
(86) PCT No.: PCT/CN2020/092222

§ 371 Date: Dec. 24, 2021

(30) Foreign Application Priority Data
Jun. 24, 2019 (CN) 201910549737.1

(51) Int. Cl. A61B 1/00

(52) U.S. Cl. CPC A61B 1/00154; A61B 2560/0406; A61B 1/0008

(57) ABSTRACT

An endoscope forceps elevator. The bottom end of the endoscope forceps elevator communicates with an instrument channel and is provided with a hinge part that is hinged to a distal end base, and the top end thereof is provided with a positioning bayonet; and a guiding groove is formed between the instrument channel and the positioning bayonet. Before the endoscope forceps elevator being lifted, an instrument to be positioned may be guided into the positioning bayonet by the guiding groove and be laterally spaced and positioned. In the endoscope forceps elevator, the instrument to be positioned may be directly positioned by the positioning bayonet, the instrument to be positioned, such as a catheter or guide wire, may be precisely and effectively positioned during the process of lifting of the forceps elevator and during a surgery. Also disclosed are a corresponding endoscope distal end and duedenoscopy system.

The present disclosure claims priority of Chinese Patent Application No. 201910549737.1, titled “ENDOSCOPE FORCEPS LIFTING DEVICE, ENDOSCOPE HEAD PART AND DUODENOSCOPE SYSTEM”, filed with the China National Intellectual Property Administration on Jun. 24, 2019, which is incorporated herein by reference in its entirety.

### FIELD

The present disclosure relates to the technical field of medical instruments, and in particular to an endoscopic forceps elevator, an endoscopic distal end, and a duodenoscopic system.

### BACKGROUND

Cancer is a general term for a major malignant tumor. Cancer cells have a character of unlimited and endless proliferation, which causes a large amount of nutrients in a body of patient to be consumed. China is a major country prone to gastrointestinal diseases, and has been in trouble with a low detection rate and a high mortality of gastrointestinal diseases for a long time. At present, the most effective way to fight cancer is recognized internationally as early detection, early diagnosis, and early treatment. Early cancer diagnosis is a diagnosis and treatment method specifically for early cancer patients. Endoscopic diagnosis is the most representative early diagnosis of gastrointestinal cancer, which has an advantage of observing and taking pictures at the same time, and is widely used in clinical practice.

A duodenoscopy refers to a method that uses a duodenoscope to perform duodenoscope diagnosis, which can be applied to diagnose duodenal, liver, gallbladder or pancreatic diseases, and can also be applied to adjuvant treatment of the digestive system. When performing a treatment of the digestive system or pancreaticobiliary system by means of a duodenoscope, in addition to the use of endoscope to perform the treatment of angiographic diagnosis to the digestive tract, bile duct or pancreatic duct, there is further a treatment of recovering gallstones presented in the common bile duct by balloons or holding treatment instrument.

ERCP is endoscopic retrograde cholangiopancreatography. During the ERCP operation, the duodenoscope is inserted into a descending portion of the duodenum to find the duodenal papilla, and a catheter is inserted via the inside of the instrument channel to the papilla opening, and then a contrast agent is injected to take X-rays images so as to show a pancreaticobiliary duct, and surgical operations such as incision and stone recovery are performed on the pancreaticobiliary duct.

When a treatment instrument needs to be replaced during the ERCP operation, a guide wire is inserted inside the treatment instrument, a distal portion of the guide wire remains of being inserted into the papilla, and a subsequent treatment instrument is guided by the guide wire to be inserted into the papilla.

A forceps elevator is arranged at an endoscopic distal end. Extension angle of the catheter or other treatment instruments can be controlled by lifting and lowering of the forceps elevator, and the catheter or other treatment instruments can be inserted into the papilla to reach the pancreatic duct or bile duct by cooperating with the bending and rotation of an endoscope.

In the conventional technologies, a technical problem to be urgently solved by those skilled in the art is how to effectively and accurately position and fix the catheter, guide wire and other treatment instruments, so as to prevent the guide wire and other treatment instruments from coming out of the papilla and being moved to the inside of the papilla.

### SUMMARY

In view of this, an object of the present disclosure is to provide an endoscopic forceps elevator, an endoscopic distal end and a duodenoscopic system, so as to prevent a guide wire from coming out of a papilla and moving to the inside of the papilla, so that the effective fixation and precise adjustment to a treatment instrument is realized, thereby reducing the risk of surgery and increasing the convenience of usage.

In order to achieve the above objects, the following technical solutions are provided according to the present disclosure.

An endoscopic forceps elevator, where the bottom end of the endoscopic forceps elevator is in communication with an instrument channel, and a hinge portion for hinging with a distal end base is arranged at the bottom end of the endoscopic forceps elevator;
a positioning bayonet configured to guide an extension direction of an instrument to be positioned is arranged at a top end of the endoscopic forceps elevator, and a guiding groove is provided between the instrument channel and the positioning bayonet; two ends of the guiding groove are in communication with the instrument channel and the positioning bayonet, respectively, and before the endoscopic forceps elevator is lifted, the instrument to be positioned is guided into the positioning bayonet and position-limited laterally by the guiding groove.

Preferably, a holding groove is provided between the positioning bayonet and an upper portion of the guiding groove, and the holding groove is arranged inwardly along the positioning bayonet; a front edge of the holding groove is a first edge of the positioning bayonet, and a clamping portion is formed at a junction of a rear edge of the holding groove with a recess portion of the forceps elevator; after the endoscopic forceps elevator is lifted, the clamping portion is configured to cooperate with an abutting and fixing member to clamp and fix the instrument to be positioned.

Preferably, the holding groove is in a strip-shaped groove structure.

Preferably, a junction edge is formed at a junction of one side edge of the holding groove with the guiding groove, and during the forceps elevator being lifted, the instrument to be positioned is slid into the holding groove along the junction edge.

Preferably, before the endoscopic forceps elevator is lifted, in a process that the instrument to be positioned is guided by the guiding groove to extend out of the positioning bayonet through the guiding groove, an extending direction of an end of the instrument to be positioned is deflected, and a deflection angle ranges from 30 degrees to 90 degrees.

Preferably, from a time when the instrument to be positioned just extends into the guiding groove through the instrument channel, to a time when the forceps elevator is controlled to be lifted and the instrument to be positioned is clamped and fixed, and a turning angle of the instrument to be positioned is greater than 90 degrees.

Preferably, for the above endoscopic forceps elevator, the instrument to be positioned is a catheter, a guide wire or other treatment instruments.

Preferably, the cross-sectional profile of the holding groove is U-shaped, V-shaped, T-shaped, or arc-shaped.

Preferably, for the above endoscopic forceps elevator, the diameter of a bottom arc of the positioning bayonet is 1 mm, so that the guide wire comes into the positioning bayonet.

An endoscopic distal end includes a distal end base and a forceps elevator, where the forceps elevator is the endoscopic forceps elevator described above;
the distal end base is provided with an accommodating space for accommodating the forceps elevator, an abutting and fixing member is arranged in the accommodating space, and a positioning surface is defined on the abutting and fixing member;
a hinge portion of the forceps elevator is hinged with the distal end base by a pivotal arm; after the forceps elevator is lifted, the forceps elevator is configured to cooperate with the positioning surface to clamp and fix the instrument to be positioned.

Preferably, the positioning surface is a flat surface, an arc-shaped surface or a stepped surface, and the positioning surface is configured to penetrate into a clamping portion of a holding groove of the forceps elevator to form a clamping structure therewith.

Preferably, a clamping gap of the clamping structure is less than or equal to 0.2 mm.

Preferably, the accommodating space may be in a through-hole structure, a blind-hole structure, other regular-shaped through-slot structures or irregular-shaped through-slot structures.

A duodenoscopic system, where an endoscope of the duodenoscopic system is provided with the endoscopic distal end described above;
the distal end base in the endoscopic distal end is further provided with a nozzle of a water vapor system, an objective lens of an imaging system and a lens of an illumination system;
a distal end cap is covered on the outside of the distal end base, and the distal end cap is detachably connected to the distal end base.

The endoscopic forceps elevator, endoscopic distal end and duodenoscopic system provided according to the present disclosure enable the guide wire to be directly positioned by the positioning bayonet, so that both in the subsequent lifting process of the forceps elevator and the surgery process, the guide wire can be accurately and effectively positioned to prevent the guide wire from sliding laterally and deflecting from the clamping position, which facilitate the effective fixation and precise adjustment to the treatment instrument, so as to prevent the guide wire from coming out of the papilla and moving to the inside of the papilla, thereby reducing the risk of surgery and increasing the convenience of usage.

Moreover, even in a case that the instrument to be positioned such as a guide wire is excessively bent or is relative thin and soft, resulting in a small resilience, since a clamping gap is formed by the clamping surface and the clamping portion, the instrument to be positioned such as the guide wire can be hold in effectively fixation by the clamping gap.

### BRIEF DESCRIPTION OF THE DRAWINGS

For more clearly illustrating embodiments of the present disclosure or the technical solutions in the conventional technology, drawings referred to for describing the embodiments or the conventional technology will be briefly described hereinafter. Apparently, drawings in the following description are only examples of the present disclosure, and for the person skilled in the art, other drawings may be obtained based on the provided drawings without any creative efforts.

FIG. 1 is a schematic structural view of an endoscopic forceps elevator provided according to a first specific embodiment of the present disclosure;

FIG. 2 is a schematic trajectory view of an instrument to be positioned moving in a guiding groove of the endoscopic forceps elevator provided according to the first specific embodiment of the present disclosure;

FIG. 3 is a schematic view of a positional relation between a guide wire and a treatment instrument when the guide wire of the endoscopic forceps elevator is positioned in a positioning bayonet provided according to the first specific embodiment of the present disclosure;

FIG. 4 is a deflected trajectory view of the guide wire in a process of extending out from the endoscopic forceps elevator provided according to the first specific embodiment of the present disclosure;

FIG. 5 is a schematic view of a positional relation between the endoscopic forceps elevator and an abutting and fixing member before the endoscopic forceps elevator provided according to the first specific embodiment of the present disclosure is lifted;

FIG. 6 is a partial cross-sectional view of an endoscopic lens when the endoscopic forceps elevator cooperating with the abutting and fixing member to clamp the guide wire after the endoscopic forceps elevator provided according to the first embodiment of the present disclosure is lifted;

FIG. 7 is a schematic structural view of when a clamping portion of the endoscopic forceps elevator and a positioning surface of the abutting and fixing member are cooperated to form a clamping gap provided according to the first specific embodiment of the present disclosure;

FIG. 8 is a schematic structural view of an abutting and fixing member provided according to a second specific embodiment of the present disclosure;

FIG. 9 is a schematic view of an internal structure of an endoscopic distal end provided according to the second embodiment of the present disclosure;

FIG. 10 is a schematic structural view of the guide wire extending out from the endoscopic distal end before the forceps elevator provided according to the second embodiment of the present disclosure is lifted; and

FIG. 11 is a schematic structural view of a duodenoscopic system provided according to a third embodiment of the present disclosure.

### REFERENCE NUMERALS IN FIGURES

distal end base 1; forceps elevator 2; abutting and fixing member 3; guide wire 4; other treatment instruments (treatment instruments except the guide wire 4) 5; pivotal arm 11; traction wire 12; nozzle 13; objective lens 14; lens 15; positioning bayonet 20; guiding groove 21; clamping groove 22; clamping portion 23; first edge 211; second edge 212; junction edge 213; positioning surface 30; light source apparatus 100; processor apparatus 200; treatment insertion portion 300; operation end 301; endoscopic distal end 302.

### DETAILED DESCRIPTION OF THE EMBODIMENTS

The technical solutions according to the embodiments of the present application will be described clearly and completely as follows in conjunction with the drawings in the embodiments of the present application. It is apparent that the described embodiments are only a part of the embodiments according to the present application, rather than all of the embodiments. Based on the embodiments of the present application, all other embodiments obtained without creative efforts by those of ordinary skill in the art shall fall within the protection scope of the present application.

### First Specific Embodiment

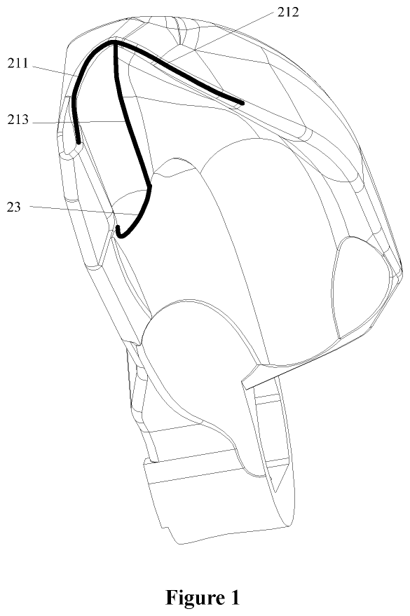

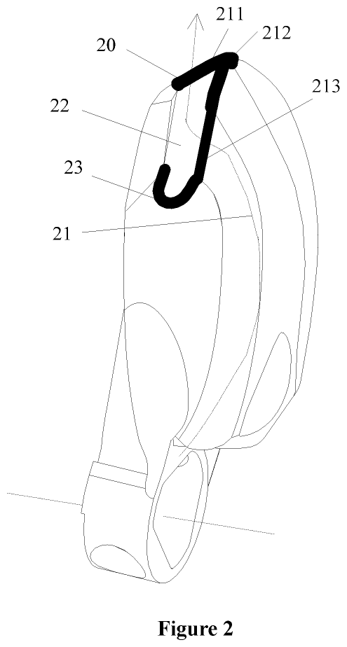

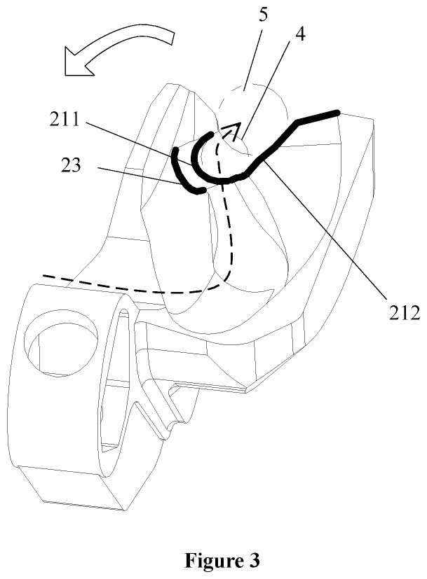

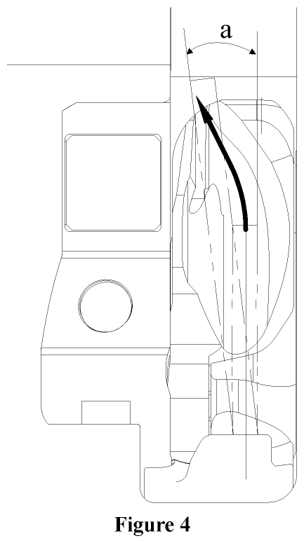

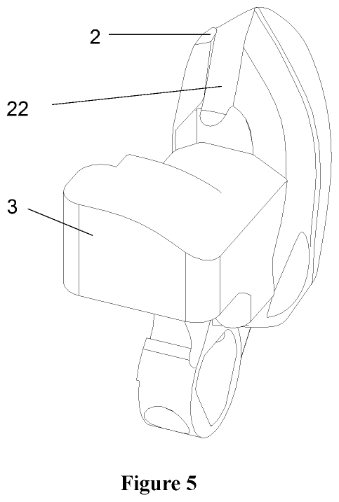

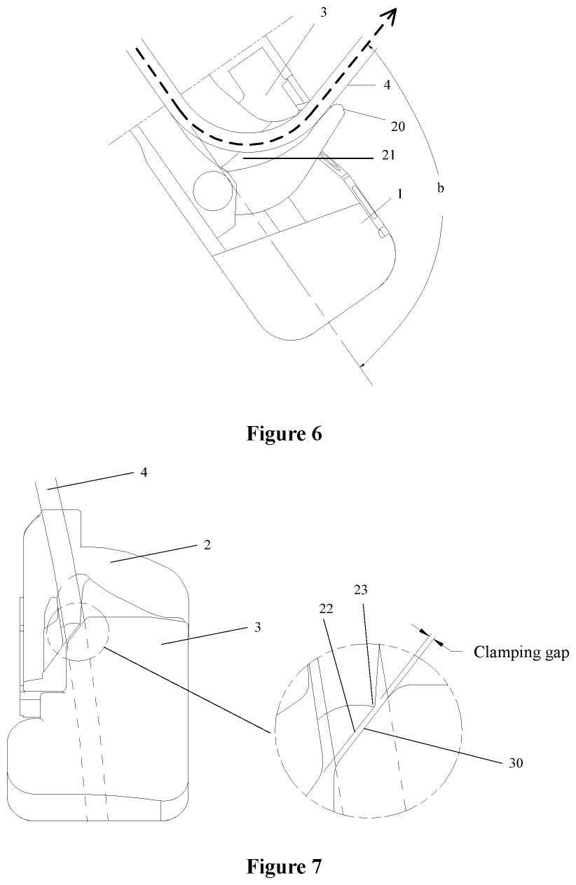

Reference is made to FIGS. 1 to 7. FIG. 1 is a schematic structural view of an endoscopic forceps elevator provided according to a first specific embodiment of the present disclosure; FIG. 2 is a schematic trajectory view of an instrument to be positioned moving in a guiding groove of the endoscopic forceps elevator provided according to the first specific embodiment of the present disclosure; FIG. 3 is a schematic view of a positional relation between a guide wire and a treatment instrument when the guide wire of the endoscopic forceps elevator is positioned in a positioning bayonet provided according to the first specific embodiment of the present disclosure; FIG. 4 is a deflected trajectory view of the guide wire in a process of extending out from the endoscopic forceps elevator provided according to the first specific embodiment of the present disclosure; FIG. 5 is a schematic view of a positional relation between the endoscopic forceps elevator and an abutting and fixing member before the endoscopic forceps elevator provided according to the first specific embodiment of the present disclosure is lifted; FIG. 6 is a partial cross-sectional view of an endoscopic lens when the endoscopic forceps elevator cooperating with the abutting and fixing member to clamp the guide wire after the forceps is lifted by the endoscopic forceps elevator provided according to the first embodiment of the present disclosure is lifted; FIG. 7 is a schematic structural view of when a clamping portion of the endoscopic forceps elevator and a positioning surface of the abutting and fixing member are cooperated to form a clamping gap provided according to the first specific embodiment of the present disclosure.

An endoscopic forceps elevator (referred to as forceps elevator for short) is provided according to the first specific embodiment of the present disclosure, where the bottom end of the forceps elevator is in communication with an instrument channel, and a hinge portion for being hinged with a distal end base 1 is arranged at the bottom end of the forceps elevator; a positioning bayonet 20 configured to guide an extension direction of an instrument to be positioned is arranged at a top end of the forceps elevator; after the forceps elevator is lifted, the positioning bayonet 20 is configured to cooperate with an abutting and fixing member 3 to guide and position the instrument to be positioned; the positioning bayonet 20 includes a first edge 211 and a second edge 212, which are connected to form the positioning bayonet (the first edge 211 and the second edge 212 are both shown in bold in FIG. 1, FIG. 2 and FIG. 3).

As shown in FIG. 1, FIG. 2 and FIG. 3, a guiding groove 21 is provided between the instrument channel and the positioning bayonet 20, and two ends of the guiding groove 21 are in communication with the instrument channel and the positioning bayonet 20, respectively. Before the forceps elevator is lifted, the instrument to be positioned is guided into the positioning bayonet 20 and position-limited laterally by the guiding groove 21.

It should be noted here that, “before the forceps elevator is lifted” includes a state before the forceps elevator forceps is not lifted at all, and also includes a state that the forceps elevator is lifted at a certain angle without clamping the instrument to be positioned, so that the instrument to be positioned can extend out from the forceps elevator; “being position-limited laterally” means that a side wall of the instrument to be positioned is clamped (or blocked or stopped) by the positioning bayonet 20 to prevent the instrument to be positioned from sliding laterally and coming out from the guiding position.

As shown in FIGS. 2 and 3, between the positioning bayonet 20 and the guiding groove 21, a holding groove 22 is provided at the upper portion of the guiding groove 21, where the holding groove 22 is a strip-shaped groove structure. The holding groove 22 is arranged inwardly along the positioning bayonet 20, a front edge of the holding groove 22 is a first edge 211 of the positioning bayonet 20 to form a notch of the holding groove 22, where the notch is configured to guide and position the instrument to be positioned. A junction edge 213 is formed at a junction of one side edge of the holding groove 22 with the guiding groove 21, and during the forceps elevator being lifted, the instrument to be positioned is slidable into the holding groove 22 along the junction edge 213. A clamping portion 23 is formed at a junction of a rear edge of the holding groove 22 with a recess portion of the forceps elevator, and the clamping portion 23 is configured to form a clamping structure with the abutting and fixing member 3, so as to clamp the instrument to be positioned.

Regarding the guiding action of the guiding groove 21, as shown in FIG. 4, the arrow direction represents a schematic view of a moving direction of the instrument to be positioned being guided to the positioning bayonet 20 by the guiding groove 21 and extending out of the forceps elevator, before the forceps elevator is lifted.

After the forceps elevator is lifted, since the instrument to be positioned such as the guide wire has a certain resilience, the instrument to be positioned will be slid to the position of the forceps elevator shown in a dashed line on the left along the junction edge 213 during a process of the forceps being lifted, so that the instrument to be positioned is held in the holding groove 22, and then is clamped by the clamping structure formed by the clamping portion 23 of the holding groove 22 and the abutting and fixing member 3, so that the instrument to be positioned is fixed. Due to the lateral position-limiting effect by the positioning bayonet 20, in a process that the instrument to be positioned is slid to the holding groove 22 along the junction edge 213, a leading-out position and a leading-out direction of the instrument to be positioned remain unchanged. Due to the holding effect of the holding groove 22 on the instrument to be positioned, the clamping position of the instrument to be positioned is predetermined, the guide wire is prevented from being deflected, and the clamping effect is better.

Specifically, the cross-sectional profile of the holding groove 22 is U-shaped, V-shaped, T-shaped, arc-shaped, or other structures.

As shown in FIG. 5, since the maximum outer diameter of the guide wire 4 is 0.97 mm, the diameter of a bottom arc of the positioning bayonet 20 is 1 mm. Therefore, the guide wire 4 can come into a recess arc of the positioning bayonet 20. That is, the guide wire 4 can be led out from the first edge 211 and positioned by the first edge 211; and the diameter or width of the other treatment instruments 5 is generally greater than 1 mm, so that the other treatment instruments 5 can be led out along the second edge 212 of the positioning bayonet 20 and is clamped and positioned by the second edge 212.

It should be noted here that the “instrument to be positioned” referred to herein is specifically a catheter, a guide wire or other treatment instruments (the treatment instruments may also be referred to as surgical instruments). In order to facilitate the description and understanding, the guide wire is taken as an example herein for specific description.

Specifically, a process of inserting the guide wire into the papilla and being fixed during a surgical operation is as follows.

Firstly, the guide wire extends into the guiding groove 21 of the forceps elevator along the instrument channel arranged in an endoscopic insertion portion.

The guide wire is continue to be inserted, as shown by the arrow in FIG. 2, the guide wire extends into the holding groove 22 along the guiding groove 21. The direction of the guide wire is laterally limited by a side wall of the holding groove 22, so that the guide wire is guided to extend into the positioning bayonet 20. Due to the position-limiting and positioning effect of the positioning bayonet 20, the direction in which the guide wire extends out of the forceps elevator is in the predetermined direction, and the positioning bayonet 20 is configured to laterally position-limit and position the guide wire.

Subsequently, the forceps elevator is controlled to be lifted (i.e., the forceps elevator is controlled to rotate around a hinge axis, and reference can be made to the direction of the big arrow in FIG. 3 for the rotation direction). With the process of forceps elevator being lifted, the instrument to be positioned may be slid into the holding groove 22 along the junction edge 213, and then the guide wire is clamped by the clamping structure formed by the clamping portion 23 of the holding groove 22 and the positioning surface 30 of the abutting and fixing member 3 (refers to FIGS. 6 and 7). In a process that the instrument to be positioned is slid to the holding groove 22 via the junction edge 213, due to the position-limiting and positioning effort of the positioning bayonet 20, the leading-out position and the leading-out direction of the instrument to be positioned remain unchanged, so that the guide wire will not come out of the papilla and not move to the inside of the papilla, which facilitates the treatment instrument 5 of being fixed, and being adjusted and positioned precisely.

Next, the extension angle of the guide wire and other treatment instruments can be controlled by controlling the lifting and lowering of the forceps elevator, and further the guide wire and other treatment instruments can be inserted into the papilla to reach the pancreatic duct or bile duct by cooperating with the bending and rotation of the endoscope.

In the above process, due to the holding effect of the holding groove 22, the guide wire 4 will not sway and deflect after being clamped and fixed.

As can be seen, the endoscopic forceps elevator provided according to the first specific embodiment of the present disclosure enables the instrument to be positioned such as the guide wire to be directly positioned by the positioning bayonet 20, so that both in the process of the forceps elevator being lifted and the surgery process performed subsequently, the guide wire can be accurately and effectively positioned to prevent the guide wire from sliding laterally and deflecting from the clamping position, thereby facilitating of achieving effective fixation and precise adjustment to the treatment instruments, so that the guide wire will not come out of the papilla and not move to the inside of the papilla, and thus it reduces the risk of surgery and increasing the convenience of usage.

Specifically, before the endoscopic forceps elevator is lifted, in a process that the guide wire is guided by the guiding groove 21 to extend out of the forceps elevator through the positioning bayonet 20, the extending direction of an end of the guide wire is deflected, and a deflection angle ranges from 30 degrees to 90 degrees. Therefore, the guiding trajectory of the guiding groove 21 is specifically a V-shaped, U-shaped or other similar structures (the “deflection angle” referred to in this paragraph is the angle a in FIG. 4).

Specifically, in the process, which is from the time when the guide wire just extends into the guiding groove 21 through the instrument channel to the time when the forceps elevator is controlled to be lifted and to clamp and fix the guide wire (at this moment, the guide wire is clamped and fixed in the clamping structure formed by the clamping portion 23 and the positioning surface 30), a turning angle of the guide wire is greater than 90 degrees (the “turning angle” referred to in this paragraph is the angle b in FIG. 6).

### Second Specific Embodiment

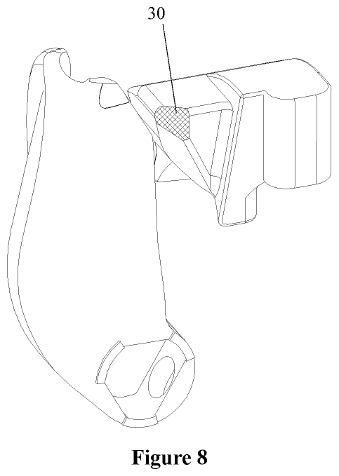

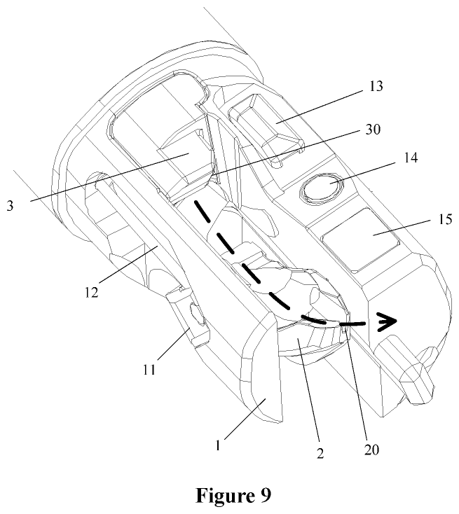

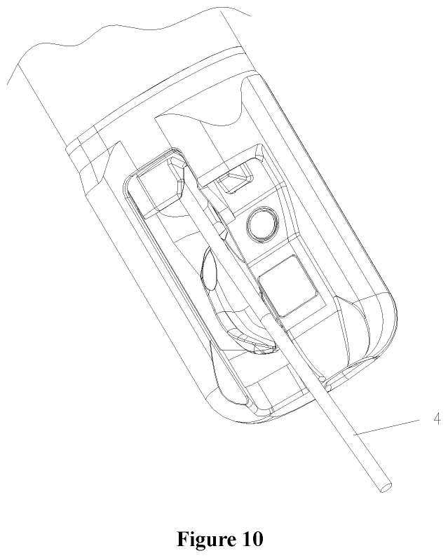

Reference is made to FIGS. 8 to 10. FIG. 8 is a schematic structural view of an abutting and fixing member provided according to a second specific embodiment of the present disclosure; FIG. 9 is a schematic view of an internal structure of an endoscopic distal end provided according to the second embodiment of the present disclosure; FIG. 10 is a schematic structural view of the guide wire extending out from the endoscopic distal end before the forceps elevator provided according to the second embodiment of the present disclosure is lifted.

The second embodiment of the present disclosure provides an endoscopic distal end, which includes a distal end base 1 and a forceps elevator 2, where the forceps elevator 2 is the endoscopic forceps elevator provided according to the first embodiment of the present disclosure.

Specifically, in the above endoscopic distal end, the distal end base 1 is provided with an accommodating space for accommodating the forceps elevator 2, an abutting and fixing member 3 is arranged in the accommodating space, and a positioning surface 30 is defined on the abutting and fixing member 3. A hinge portion of the forceps elevator 2 is hinged with the distal end base 1 by a pivotal arm 11, after the forceps elevator 2 is lifted, the instrument to be positioned is clamped and fixed by the clamping structure formed by the clamping portion 23 of the holding groove 22 and the positioning surface 30 of the abutting and fixing member 3.

Specifically, the “accommodating space” described herein may be in a through-hole structure, a blind-hole structure, or other regular or irregular-shaped groove structures, or the “accommodating space” may also be a recess space structure at the top end or side end of the distal end base. The specific structure of the distal end base and its “accommodating space” are not specifically limited hereto in the present disclosure, as long as the forceps elevator 2 can be hinged with the distal end base and relative rotation of the forceps elevator 2 is allowed, those skilled in the art can implement specific embodiments according to actual requirements.

Preferably, as shown in FIG. 9, the above “accommodating space” is in a through-hole structure or a blind hole structure or a through-groove structure, and the positioning surface 30 is located in the accommodating space and close to an opening of the instrument channel.

Specifically, the above positioning surface 30 is a flat surface, an arc-shaped surface or a stepped surface, the positioning surface 30 is configured to penetrate into the clamping portion 23 of the holding groove 22 of the forceps elevator 2 to form a clamping structure therewith.

Specifically, a clamping gap of the above clamping structure is less than or equal to 0.2 mm to ensure that the thinnest guide wire (with a diameter of 0.2 mm) can be effectively clamped (reference may be made to FIG. 7 for the “clamping gap”).

Specifically, as shown in FIG. 9, the pivotal arm 11 is arranged at a hinged end of the forceps elevator 2, and the pivotal arm 11 is driven to rotate by a traction wire 12. The traction wire 12 is preferably a steel wire. During operation, a shifter lever arranged on an operation end connected to an endoscope in the endoscopic system is manipulated to control the lifting and lowering of the forceps elevator 2 via the traction steel wire 12 and the pivotal arm 11, so that the instrument to be positioned such as the guide wire is controlled to be clamped and fixed or loosened and disassembled by the clamping portion 23 of the holding groove 22 and the positioning surface 30.

### Third Specific Embodiment

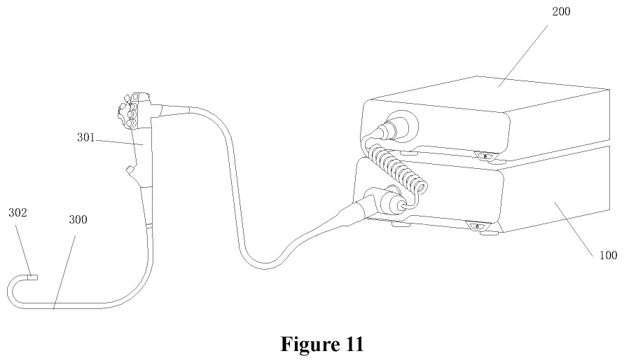

Reference is made to FIG. 11, FIG. 11 is a schematic structural view of a duodenoscopic system provided according to a third embodiment of the present disclosure.

A duodenoscopic system is provided according to the third specific embodiment of the present disclosure, an endoscope of the duodenoscopic system is provided with the endoscopic distal end provided according to the first specific embodiment of the present disclosure.

The duodenoscopic system includes an endoscope, a light source portion 100 and a host portion 200, where the endoscope generally includes an insertion portion 300 for inserting into a body cavity to be checked, and one end of the insertion portion 300 is a distal end 302, the other end is an operation end 301. The distal end 302 is provided with the forceps elevator 2, and the operation end 301 is provided with a shifter lever for controlling the lifting and lowering of the forceps elevator 2. The light source portion 100 is configured to provide white light illumination and special light illumination for the endoscope, and the host portion 200 is configured to provide signal control for the endoscope and to process images captured by the endoscope, where the host portion 200 is connected to a display to realize the display of endoscopic images.

Specifically, the distal end base in the above endoscope is further provided with a nozzle 13 of a water vapor system, an objective lens 14 of an imaging system and a lens 15 of an illumination system, where the nozzle 13 is configured to spray fluids such as water or air onto an outer surface of the objective lens 15 to clean the objective lens 15. An imaging element CCD of an observation optical system is arranged on an inner side of the objective lens of the imaging system, which is configured to send the image information captured by the insertion portion back to the host portion for display. The lens 15 is connected to an optical fiber served as a light transmission path.

Further, a distal end cap is covered on the outside of the distal end base of the clamping structure of the endoscope described above, and the distal end cap is detachably connected to the distal end base 1.

Finally, it should be further illustrated that a relation term such as “first” and “second” herein is only used to distinguish one entity or operation from another entity or operation, and does not necessarily require or imply that there is an actual relation or sequence between these entities or operations. Moreover, the terms “comprise”, “include”, or any other variants thereof are intended to encompass a non-exclusive inclusion, such that the process, method, article, or device including a series of elements includes not only those elements but also those elements that are not explicitly listed, or the elements that are inherent to such process, method, article, or device. Unless explicitly limited, the statement “including a . . . ” does not exclude the case that other similar elements may exist in the process, the method, the article or the device other than enumerated elements.

The above embodiments are described in a progressive manner. Each of the embodiments is mainly focused on describing its differences from other embodiments, and reference may be made among these embodiments with respect to the same or similar parts.

The above illustration of the disclosed embodiments can enable those skilled in the art to implement or use the present application. Various modifications to the embodiments are apparent to the person skilled in the art, and the general principle herein can be implemented in other embodiments without departing from the spirit or scope of the present application. Therefore, the present application is not limited to the embodiments described herein, but should be in accordance with the broadest scope consistent with the principle and novel features disclosed herein.

1. An endoscopic forceps elevator, wherein a bottom end of the endoscopic forceps elevator is in communication with an instrument channel, and a hinge portion for hinging with a distal end base is arranged at the bottom end of the endoscopic forceps elevator;
a positioning bayonet configured to guide an extension direction of an instrument to be positioned is arranged at a top end of the endoscopic forceps elevator, and a guiding groove is provided between the instrument channel and the positioning bayonet; two ends of the guiding groove are in communication with the instrument channel and the positioning bayonet, respectively, and before the endoscopic forceps elevator is lifted, the instrument to be positioned is able to be guided into the positioning bayonet and position-limited laterally by the guiding groove.

2. The endoscopic forceps elevator according to claim 1, wherein a holding groove is provided between the positioning bayonet and an upper portion of the guiding groove, and the holding groove is arranged inwardly along the positioning bayonet; a front edge of the holding groove is a first edge of the positioning bayonet, and a clamping portion is formed at a junction of a rear edge of the holding groove with a recess portion of the forceps elevator; after the endoscopic forceps elevator is lifted, the clamping portion is configured to cooperate with an abutting and fixing member to clamp and fix the instrument to be positioned.

3. The endoscopic forceps elevator according to claim 2, wherein the holding groove is in a strip-shaped groove structure.

4. The endoscopic forceps elevator according to claim 2, wherein a junction edge is formed at a junction of one side edge of the holding groove with the guiding groove, and during the forceps elevator being lifted, the instrument to be positioned is slid into the holding groove along the junction edge.

5. The endoscopic forceps elevator according to claim 12, wherein before the endoscopic forceps elevator is lifted, in a process that the instrument to be positioned is guided by the guiding groove to extend out of the positioning bayonet through the guiding groove, an extending direction of an end of the instrument to be positioned is deflected, and a deflection angle ranges from 30 degrees to 90 degrees.

6. The endoscopic forceps elevator according to claim 12, wherein from a time when the instrument to be positioned just extends into the guiding groove through the instrument channel, to a time when the forceps elevator is controlled to be lifted and the instrument to be positioned is clamped and fixed, a turning angle of the instrument to be positioned is greater than 90 degrees.

7. The endoscopic forceps elevator according to claim 1, wherein the instrument to be positioned is a catheter or a guide wire.

8. The endoscopic forceps elevator according to claim 2, wherein the cross-sectional profile of the holding groove is U-shaped, V-shaped, T-shaped, or arc-shaped.

9. The endoscopic forceps elevator according to claim 1, wherein the diameter of a bottom arc of the positioning bayonet is 1 mm, so that a guide wire is able to come into the positioning bayonet.

10. An endoscopic distal end, comprising a distal end base and a forceps elevator, wherein the forceps elevator is the endoscopic forceps elevator according to claim 1;
the hinge portion of the forceps elevator is hinged with the distal end base by a pivotal arm;
the distal end base is provided with an accommodating space for accommodating the forceps elevator, an abutting and fixing member is arranged in the accommodating space, and a positioning surface is defined on the abutting and fixing member;
after being lifted, the forceps elevator is configured to cooperate with the positioning surface to clamp and fix the instrument to be positioned.

11. The endoscopic distal end according to claim 10, wherein a holding groove is provided between the positioning bayonet and an upper portion of the guiding groove, and the holding groove is arranged inwardly along the positioning bayonet a front edge of the holding groove is a first edge of the positioning bayonet, and a clamping portion is formed at a junction of a rear edge of the holding groove with a recess portion of the forceps elevator;
the positioning surface is a flat surface, an arc-shaped surface or a stepped surface, and the positioning surface is configured to penetrate into the clamping portion of a holding groove of the forceps elevator to form a clamping structure with the clamping portion, and the clamping structure is configured to clamp and fix the instrument to be positioned.

12. The endoscopic distal end according to claim 11, wherein a clamping gap of the clamping structure is less than or equal to 0.2 mm.

13. The endoscopic distal end according to claim 10, wherein the accommodating space is in a through hole structure, a blind hole structure or a through groove structure.

14. A duodenoscopic system, wherein an endoscope of the duodenoscopic system is provided with the endoscopic distal end according to claim 10;
the distal end base in the endoscopic distal end is further provided with a nozzle of a water vapor system, an objective lens of an imaging system and a lens of an illumination system;
a distal end cap is covered on the outside of the distal end base, and the distal end cap is detachably connected to the distal end base.

15. The endoscopic distal end according to claim 10, wherein a holding groove is provided between the positioning bayonet and an upper portion of the guiding groove, and the holding groove is arranged inwardly along the positioning bayonet; a front edge of the holding groove is a first edge of the positioning bayonet, and a clamping portion is formed at a junction of a rear edge of the holding groove with a recess portion of the forceps elevator; after the endoscopic forceps elevator is lifted, the clamping portion is configured to cooperate with an abutting and fixing member to clamp and fix the instrument to be positioned

16. The endoscopic distal end according to claim 10, wherein before the endoscopic forceps elevator is lifted, in a process that the instrument to be positioned is guided by the guiding groove to extend out of the positioning bayonet through the guiding groove, an extending direction of an end of the instrument to be positioned is deflected, and a deflection angle ranges from 30 degrees to 90 degrees.

17. The endoscopic distal end according to claim 10, wherein from a time when the instrument to be positioned just extends into the guiding groove through the instrument channel, to a time when the forceps elevator is controlled to be lifted and the instrument to be positioned is clamped and fixed, a turning angle of the instrument to be positioned is greater than 90 degrees.

18. The endoscopic distal end according to claim 10, wherein the diameter of a bottom arc of the positioning bayonet is 1 mm, so that a guide wire is able to come into the positioning bayonet.

19. The duodenoscopic system according to claim 14, wherein a holding groove is provided between the positioning bayonet and an upper portion of the guiding groove, and the holding groove is arranged inwardly along the positioning bayonet; a clamping portion is formed at a junction of a rear edge of the holding groove with a recess portion of the forceps elevator;
the positioning surface is configured to penetrate into the clamping portion to form a clamping structure with the clamping portion, and the clamping structure is configured to clamp and fix the instrument to be positioned.

20. The duodenoscopic system according to claim 14, wherein before the endoscopic forceps elevator is lifted, in a process that the instrument to be positioned is guided by the guiding groove to extend out of the positioning bayonet through the guiding groove, an extending direction of an end of the instrument to be positioned is deflected, and a deflection angle ranges from 30 degrees to 90 degrees.
